(19) United States
(12) Patent Application Publication
(10) Pub. No.: US 2025/0222207 A1
(43) Pub. Date: Jul. 10, 2025

(54) Injection Device Comprising a Setting Dose Member

(71) Applicant: Becton Dickinson France, Le Pont-de-Claix (FR)

(72) Inventors: Longxiang Huang, Suzhou (CN); Wilson Huang, Suzhou (CN); Bo Yan, Shanghai (CN)

(21) Appl. No.: 18/846,989

(22) PCT Filed: Mar. 13, 2023
(86) PCT No.: PCT/EP2023/056291

§ 371 Date: Sep. 13, 2024

(30) Foreign Application Priority Data
Mar. 15, 2022 (EP) 22305302.6

(51) Int. Cl. A61M 5/315

(52) U.S. Cl. CPC A61M 5/31551; A61M 5/31501; A61M 5/31595; A61M 2005/31508

(57) ABSTRACT

An injection device including a barrel, a plunger rod received within the barrel and including N radial projections successively disposed at different locations along the axis of the barrel, N being an integer equal or greater than 2, a dose setting member configured to be removably mounted onto the barrel, and having N portions configured to sequentially abut axially against the N radial projections, respectively, when pushing the plunger rod axially within the barrel, and the dose setting member being configured to be moved relative to the barrel between N positions, wherein the n^{th} radial projection axially faces the n^{th} portion in the n^{th} position, n being an integer lying between 1 and N.

### CROSS-REFERENCE TO RELATED APPLICATIONS

This application is the United States national phase of International Patent Application No. PCT/EP2023/056291 filed Mar. 13, 2023, and claims priority to European Patent Application No. 22305302.6 filed Mar. 15, 2022, the disclosures of which are hereby incorporated by reference in their entireties.

### BACKGROUND OF THE INVENTION

### Field of the Invention

The invention relates to an injection device, for example a medical injection device such as a syringe or the like.

### Description of Related Art

Today medical injection devices-such as prefilled syringes-intended to contain and deliver several doses of the same medical injection fluid are not always convenient for delivering accurate doses, for example small doses (e.g. 1 ml). Moreover, expelling the potential air bubble contained in the medical injection device may require a high activation force, and thus decrease the dose accuracy of the fluid to be injected. For example, the injection devices disclosed by U.S. Pat. Nos. 5,601,077, 9,579,465, US 2021/0196906 may be not so easy to handle and or not so accurate. In addition, the structure of such devices may be very complex. Therefore, a need exists to provide a reliable and user-friendly injection device for delivering accurate doses and/or showing a simple structure.

### SUMMARY OF THE INVENTION

An embodiment relates to an injection device including a barrel extending along an axis and defining a reservoir, the barrel comprising a proximal end and a distal end having an outlet, a plunger rod received within the barrel via the proximal end and configured to move along the axis relative to the barrel, the plunger rod comprising N radial projections successively disposed at different locations along the axis, N being an integer equal or greater than 2, a dose setting member configured to be mounted onto the barrel and having N portions configured to sequentially abut axially against the N radial projections, respectively, when moving the plunger rod axially in a first direction (or distal direction) oriented from the proximal end toward the distal end, the dose setting member being configured to be moved relative to the barrel between N positions, wherein the n^{th} radial projection axially faces the n^{th} portion in the n^{th} position, n being an integer lying between 1 and N.

An axial direction is the direction defined by the axis of the barrel. A radial direction is a direction perpendicular to the axis. A circumferential or azimuthal direction corresponds to the direction describing a ring around the axial direction. The three directions axial, radial and azimuthal correspond respectively to the directions defined by the height, the radius and the angle in a cylindrical coordinate system. In addition, upstream and downstream may be defined with respect to the moving direction of the plunger rod to expel/deliver the content of the reservoir, when moved in the first direction.

A distal side is the side of the injection device from which the content of the reservoir is expelled/delivered, and a proximal side is the side opposite to the distal end along the axis, which is usually handled by the user during injection.

Each n^{th} radial projection may comprise a single or several protrusions extending radially. Each n^{th} portion may comprise a single or several abutment/shoulder parts. A single abutment/shoulder part of the n^{th} portion may be configured to cooperate with a single or several protrusions of the n^{th} radial projection. Several abutment/shoulder parts of the n^{th} portion may be configured to cooperate with a single or several protrusions of the n^{th} radial projection.

The radial projection is successively disposed, i.e. arranged/ordered from the 1^{st} radial projection up to N^{th} the radial projection along the axis (when the plunger rod is mounted within the reservoir). In other words, the n^{th} radial projection is between the (n−1)^{th} radial projection and the (n+1)^{th} radial projection.

The portions may be successively arranged, but not necessarily.

The N portions are configured to sequentially abut axially against the N projections, respectively, i.e. the 1^{st} projection is configured to axially abut against the 1^{st} radial projection, the 2^{nd} projection is configured to axially abut against the 2^{nd} radial projection, etc.

For example, the movement of the dose setting member between two successive positions may be a translation, a rotation, a combination thereof, etc.

The plunger rod may comprise, 2, 3, 4, etc. radial projections and the dose setting member may comprise the same number of portions, i.e. 2, 3, 4, etc., respectively, and may be move between the same number of positions, i.e. 2, 3, 4, etc. respectively. In the 1^{st} position, the 1^{st} radial projection axially face the 1^{st} portion, in the 2^{nd} position, the 2^{nd} radial projection axially face the 2^{nd} portion, etc.

During a single total injection stroke of the plunger rod (i.e. in the first direction), the dose setting member is successively placed in the 1^{st}, 2^{nd}, 3^{rd} (if any), etc. position, so that the 1^{st}, 2^{nd}, 3^{rd} (if any), etc. radial projection successively axially face the 1^{st}, 2^{nd}, 3^{rd} (if any), etc. portion. The partial stroke from the initial position of the plunger rod up to the abutment of the 1^{st} radial projection with the 1^{st} portion correspond to a first dose (which may be a priming dose), the partial stroke from the 1^{st} radial projection up to the abutment of the 2^{nd} radial projection with the 2^{nd} portion correspond to a second dose, etc.

By moving the dose setting member between the different positions, a predetermined dose is pre-set. For example, in the n^{th} position, the injection device is pre-set for an n^{th} injection.

In other words, the dose setting member is firstly placed in the 1^{st} position. Then a first delivery (or priming) may be carried out by moving the plunger rod from its initial position (the reservoir being full, not injection/delivery carried out yet), up to abutting the 1^{st} radial projection with the 1^{st} portion. Subsequently, the dose setting member may be moved in the 2^{nd} position. Then a second delivery (or first injection) may be carried out by moving the plunger rod from its former position (corresponding to the axial position of the plunger rod at the end of the first injection/delivery, i.e. when the 1^{st} radial projection abuts the 1^{st} portion), up to abutting the 2^{nd} radial projection with the 2^{nd} portion. Etc.

Due to the configuration of the injection device, the user may easily and reliably set a dose by simply moving of the dose setting member relative to the reservoir while the axial abutment of the radial projections with the portion ensure a reliable stopping means. In addition, the configuration of the radial projections and of the portions may provide a simple structure.

In some embodiments, the injection device may be configured so that the n^{th} portion is able to abut only against the n^{th} radial projection.

In some embodiments, the dose setting member may be configured to adopt at least one N+1 position, wherein the plunger rod is axially free relative to the dose setting member.

For example, when the dose setting member is placed in the N+1 position, the plunger rod may be moved for carrying out a (N+1)^{th} injection after the N^{th} injection (when the N^{th} radial projection abuts against the N^{th} portion) or in second direction (or proximal direction), opposed to the first direction in order to suck/pump a substance to be injected, for example a liquid but not necessarily, within the reservoir, via the outlet. Alternatively the medical injection device may be prefilled with the substance to be injected. The injection device may comprise a single N+1 position or several N+1 positions, for example a first N+1 position in order to suck/pump a substance to be injected, and a second N+1 position in order to carry out a (N+1)^{th} injection. For example, the first N+1 position may be an initial position before the 1^{st} position (in such a case the first N+1 position may be also considered as a 0 position) and the second N+1 position may be the last position, after the N^{th} position.

In some embodiments, the dose setting member may be removably mounted onto the barrel.

With such a configuration, a N+1^{th} dose may be set, corresponding to the last stroke of the plunger rod from the axial position of the plunger rod at the end of the N^{th} injection, i.e. when the N^{th} radial projection abuts the N^{th} portion, up to an abutment of the plunger rod with a part of the barrel, for example a bottom of the reservoir defined in fluid communication with the outlet.

In some embodiments, the dose setting member may be configured to be radially moved relative to the barrel between N positions.

Such a radial movement may be easy to carried out, and allows a reliable checking for the user in order to be sure that the appropriate dose is set.

In some embodiments, the dose setting member may comprise a notch extending radially and radially opened, considered when the dose setting member is mounted onto the barrel, and configured to receive the barrel.

When the dose setting member is mounted onto the barrel, the notch may extend in a plane perpendicular to the axis. The walls extending around the notch or defining the notch, may define a general U-shape. The barrel and the plunger rod may be introduced within the notch via the aperture of the U-shape, i.e. the open top of the “U”, and extend transversally to the plane in which the “U-shape” extends.

In some embodiments, the notch may extend along a second axis, the N portions being successively disposed at different locations along the second axis.

When the dose setting portion is mounted onto the barrel, the second axis may extend radially. The portions may be arranged/ordered from the 1^{st} portion up to N^{th} the portion along the second axis. In other words, the n^{th} portion may be between the (n−1)^{th} portion and the (n+1)^{th} portion.

In some embodiments, a radial extension of the t^{th} radial projection may be greater than a radial extension of the (t−1)^{th} radial projection and lower than a radial extension of the (t+1)^{th} radial projection, if any, t being an integer lying between 2 and N.

In other words, the radial projections may be arranged in growing size from the 1^{st} to the N^{th} radial projection.

In some embodiment, all or part of the radial projection may have a radial extension smaller than a radius of the reservoir. This allows said radial projections to be inserted within the reservoir, into the barrel.

In some embodiments, each of the radial projections may have an annular shape having a radius, the radius of the t^{th} radial projection being greater than the radius of the (t−1)^{th} radial projection, and lower than the radius of the (t+1)^{th} radial projection, if any, and each of the portions defines an axial passage having a minimal radial length, the minimal radial length of the t^{th} portion being greater than minimal radial length of the (t−1)^{th} portion and lower than minimal radial length of the (t+1)^{th} portion, if any, t being an integer lying between 2 and N.

The annular shape of each radial projection may be continuous, i.e. ring shape, or discontinuous, e.g. a series of protrusions or ring portions arranged annularly or circumferentially. The radial projections may be arranged in growing size from the 1^{st} to the N^{th} radial projection.

The minimal radial length of the t^{th} portion may be greater than the diameter on the (t−1)^{th} radial projection and lower than the diameter of the t^{th} radial projection. The minimal radial length may correspond to the shorter distance between two facing walls delimiting the notch and extending along the second direction, at the t^{th} position.

In some embodiments, the N portions may be successively arranged and may globally have a stair shape, each portion forming a step of the stair shape.

In some embodiments, in the at least one N+1 position, the dose setting member may define an axial passage having a minimal radial length which is greater than the radius of the N^{th} radial projection.

In some embodiments, the (t−1)^{th} radial projection may be axially closer to the distal end than the th radial projection.

In some embodiments, the barrel may comprise a flange extending radially, the dose setting member being configured to be slidably mounted onto the flange.

The dose setting member may have one or several grooves receiving all or part of the flange. For example, the flange may form a rib slidably engaged within the groove(s) of the dose setting member. The flange may have any shape, e.g. annular shape, oblong or elliptical shape, rhomboid shape, square, rectangular or any polygonal shape with sharp or rounded angle, etc.

In some embodiments, the flange may comprise a guiding portion configured to slidably guide the dose setting member, and a retaining portion configured to maintain the dose setting member in any of the N positions.

The guiding portion may comprise two tongues extending radially in opposed directions. For example, when the dose setting member is mounted onto the barrel, the guiding portion may extend along the second axis of the notch and may radially guide the dose setting member in translation along the second axis.

For example, the retaining portion may cooperate by snap fitting with the dose setting member. For example, the retaining member may form a ratchet configured to be engaged with different complementary portions of the dose setting member.

In some embodiments, the injection device may be a medical syringe. The syringe may be disposable and/or prefilled.

In some embodiments, the injection device may comprise a spray nozzle, which may be removable.

In some embodiments, the injection device may comprise a vial adapter, which may be removable.

An embodiment also relates to a dose setting method for pre-setting a dose of an injection device before injection, the dose setting method comprising providing an injection device according to any one of the embodiments of the present disclosure, and moving the dose setting member in any one of the N positions so that a n^{th} radial projection axially face a n^{th} portion.

In some embodiments, the dose setting member may be moved to an n^{th} position in view of an n^{th} injection.

In some embodiments, the dose setting member may be removably mounted onto the barrel and the dose setting method may comprise removing the dose setting member for setting the last dose.

In some embodiments, the dose setting member may be configured to adopt at least one N+1 position, wherein the plunger rod is axially free relative to the dose setting member and the dose setting method may comprise, moving the dose setting member in one of the at least one N+1 position before setting the first dose for filling the injection device or after completion of the N^{th} dose to allow emptying the injection device.

The configurations according to any one of the above embodiments may take part in providing a reliable, user friendly and/or simple structure and method.

### BRIEF DESCRIPTION OF THE DRAWINGS

The terms Fig., Figs., Figure, and Figures are used interchangeably in the specification to refer to the corresponding figures in the drawings.

The disclosure and its advantages can be better understood by reading the detailed description of various embodiments given as non-limiting examples. The description refers to the accompanying sheets of figures, in which:

FIG. 1 shows an injection device;

FIG. 2 shows a top view of the dose setting member;

FIG. 3 shows a lateral view of the dose setting member;

FIG. 4 shows another view of the dose setting member;

FIGS. 5A to 5G show different steps for setting a dose with the injection device;

FIGS. 6A and 6B show cross sectional views of the injection device; in section VI of FIG. 1,

FIG. 7 shows a top view of the dose setting member according to a first variant;

FIG. 8 shows a lateral view of the dose setting member according to the first variant;

FIG. 9 shows another view of the dose setting member according to the first variant;

FIGS. 10A to 10G show different steps for setting a dose with the injection device according to the first variant;

FIGS. 11A to 11C show cross sectional views of the injection device according to the first variant, in a section similar to section VI;

FIG. 12 shows a top view of the dose setting member according to a second variant;

FIG. 13 shows a lateral view of the dose setting member according to the second variant;

FIG. 14 shows another view of the dose setting member according to the second variant;

FIGS. 15A to 15G show different steps for setting a dose with the injection device according to the first variant;

FIGS. 16A to 16D show cross sectional views of the injection device according to the second variant, in a section similar to section VI.

### DESCRIPTION OF THE INVENTION

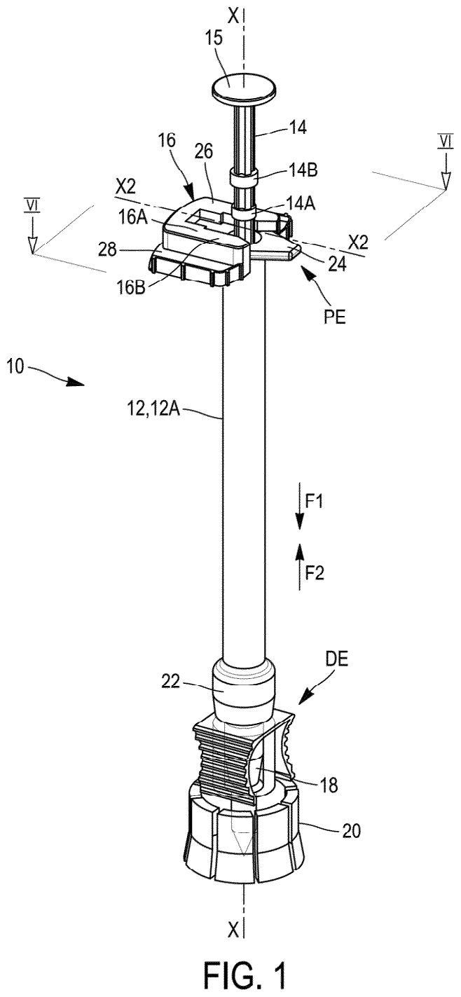

FIG. 1 show an injection device 10 including a barrel 12, a plunger rod 14 and a dose setting member 16. In the present example, the injection device 10 is a medical injection device, for example a syringe. In this example, the injection device 10 comprises spray nozzle 18 and a removable vial adapter 20. For example, the injection device 10 may be prefilled with powder drug, liquid medical component or any other active component, such as medical drug, vaccines, etc.

The barrel 12 extends along an axis X and defines a reservoir 12A therein. The barrel 12 comprises a proximal end PE and a distal end DE. The distal end DE has an outlet 22. The spray nozzle 18 and the vial adapter 20 are disposed at the distal end DE. The spray nozzle 18 is in fluid connection with the reservoir 12A via the outlet 22. Alternatively to the spray nozzle 18, a needle may be attached to the distal end DE of the barrel in order to inject the medical component to a patient.

The distal side of the injection device 10 corresponds to the side of the distal end DE and the proximal side of the injection device 10 corresponds to the side of the proximal end PE.

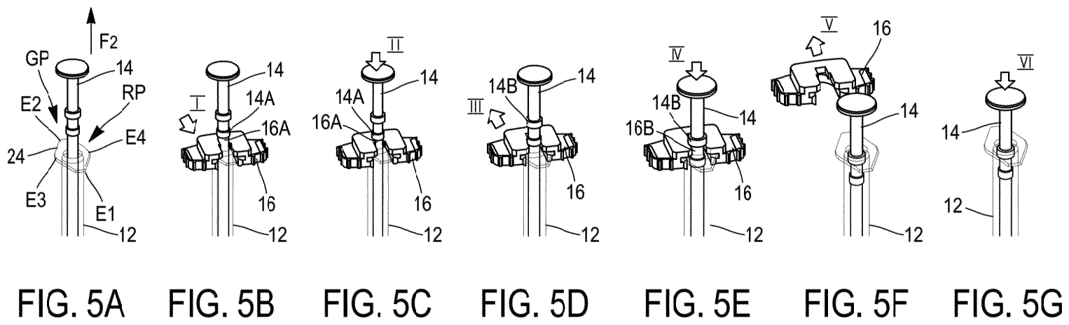

In the present example, the barrel 12 comprises a flange 24 extending radially, the flange 24 being disposed at the proximal end PE of the barrel 12. A seen in FIG. 5A, the flange 24 has a general rhomboid shape with rounded angles. In this example, the flange 24 comprises a guiding portion GP configured to slidably guide the dose setting member 16, and a retaining portion RP configured to maintain the dose setting member 16 in any of the N positions. The guiding portion GP comprises two tongues E1, E2 extending radially in opposed directions. The retaining portion RP comprises two radially opposed edges E3 and E4. The two tongues E1 and E2 extend along the larger diagonal of the rhomboid shape and the two edges E3 and E4 are disposed at the tow opposed summit joined by the smaller diagonal of the rhomboid shape. In the present example, the edges E3 and E4 from two ratchets configured to be engaged with different complementary portions of the dose setting member 10, described thereafter. In a variant, the flange may have any shape, for example a circular shape.

The plunger rod 14 is received within the barrel 12 via the proximal end PE and is configured to move along the axis X relative to the barrel 12. The plunger rod 14 comprises N radial projections 14A, 14B successively disposed at different locations along the axis X, N being an integer equal or greater than 2. In the present example N equals two (N=2). In this example, the plunger rod 14 comprises a pusher portion 15 at a proximal end thereof and a stopper (not shown) at a distal end thereof and disposed within the reservoir 3. For injection/spraying of the medical component contained in the barrel 12, the end user may apply his thumb on the pusher portion 15 of the plunger rod 14 in order to ease the injection/spraying.

In the present example, as shown in FIG. 1 and FIGS. 5A to 5G, a radial extension of the 2^{nd} radial projection 14B is greater than a radial extension of the 1^{st} radial projection 14A. In other words, a radial extension of the distal-most radial projection 14B is greater than a radial extension of the proximal-most radial projection 14A. In the present example, the radial projections 14A, 14B have an annular shape, in this example a ring shape, having a radius, the radius of the 2^{nd} radial projection 14B (or distal-most radial projection) being greater than the radius of the 1^{st} radial projection 14A (or proximal-most radial projection). The 1^{st} radial projection 14A is axially closer to the distal end DE than the 2^{nd} radial projection 14B. In the present example, the radius of all the outer radial projections is smaller than the inner radius of the reservoir 12A, so as to be able to be inserted within the reservoir 12A, into the barrel 12.

The dose setting member 16 is configured to be mounted onto the barrel 12. In this example, the dose setting member 16 is configured to be slidably mounted onto the flange 24. The dose setting member 16 has N portions 16A, 16B configured to sequentially abut axially against the N radial projections 14A, 14B, respectively, when moving the plunger rod 14 axially in a first direction (or distal direction) F1 oriented from the proximal end PE toward the distal end DE. The dose setting member 16 is configured to move relative to the barrel 12 between N positions, wherein the n^{th} radial projection axially faces the n^{th} portion in the n^{th} position, n being an integer lying between 1 and N, i.e. in the present example between 1 and 2. In the present example, the dose setting member 16 is removably mounted onto the barrel (see in particular FIGS. 5A and 5F). As shown in FIGS. 5A to 5G, the dose setting member 16 is configured to radially move relative to the barrel 12 between N positions, in a direction perpendicular to the axis X.

The dose setting member 16 comprises a notch 100 extending radially and radially opened, considered when the dose setting member 16 is mounted onto the barrel 12, and configured to receive the barrel 12. The notch 100 extends along a second axis X2, the N portions being successively disposed at different locations along the second axis X2. The walls extending around the notch 100 define a general U-shape. The barrel 12 and the plunger rod 14 may be introduced within the notch 100 via the aperture of the U-shape, i.e. the open top of the “U”, and extend transversally to the plane in which the “U-shape” extends (see FIG. 5B). When the dose setting portion 16 is mounted onto the barrel 12, the second axis X2 extends radially, perpendicularly to the axis X.

In the present example, the dose setting member 16 comprises a top portion 26, which may be a top plate, extending in a radial plane perpendicular to the axis X, considered when mounted onto the barrel 12. The top portion 26 is configured to be oriented toward the distal end of the plunger rod 14 (or in this example the flange 15 of the plunger rod 14). The lateral walls of the U-shaped notch 100 in the top portion 26 are configured to receive and cooperate with plunger rod 6. Two opposed shoulders are formed on both sides of the notch 100, at each portion 16A, 16B configured to axially abut against a radial projections 14A, 14B of the plunger rod 14. In the present example, portion 16A comprises two opposite shoulders (in this example radial shoulders) 16A1, 16A2, and portion 16B comprises two opposite shoulders (in this example radial shoulders) 16B1, 16B2. In this example, each of the portions 16A, 16B defines an axial passage having a minimal radial length, the minimal radial length RL2 of the 2^{nd} portion 16B being greater than minimal radial length RL1 of the 1^{st} portion 16A. The rest of the plunger rod 6 (i.e. the rod of the plunger rod, without the radial projections) is received within the notch 100 and is free to axially move relative to the dose setting member 16. The top portion 26 may comprises signs to indicate information about a dose at said position. In the present example, the portions 16A, 16B are successively arranged along the second axis X2, the last portion 16B being closer to the open top of the “U” than the first portion 16A.

In the present example, the dose setting member 16 comprise a lower portion 28, which may be a lower plate, extending in a radial plane perpendicular to the axis X and parallel to the second axis X2. The lower portion 28 is configured to be oriented toward the distal end DE of the barrel 12. The lateral walls of the U-shaped notch 100 in the lower portion 28 are configured to receive and cooperate with the barrel 12 (or a barrel body). The width, perpendicular to the second axis X2, of the notch 100 in the lower portion 28 is constant all along the second axis X2. This width may be greater than the maximum width of the notch 100 in the top portion 26.

The top portion 26 and the lower portion 28 are adjacent along the axis X (when mounted onto the barrel 12), and form together a same and single element. Two facing grooves 30 are arranged in the opposed lateral walls of the notch 100, axially between the top portion 26 and the lower portion 28. The grooves 30 extend along the second axis X2. An opening 38 is arranged between the two grooves 30, through which a tongue E1 or E2 of the flange 24 may extend, at least in part. The grooves 30 are configured to slidably receive at least in part the flange 24. Reliefs 32 are arranged in the grooves 30, in this example on the bottom of the grooves 30, and are configured to cooperate by snap fitting with the edges E3 and E4 of the flange 24. In other words, the reliefs 32 form different complementary portions configured to engage with the edges E3 and E4. The reliefs 32 are arranged by pair, the two members of the pair facing each other relative to the second axis X2. It the present example, each snap fitting position correspond to an n^{th} position of the dose setting member 16.

In the present example, the outer edges of the dose setting member 16 comprise a grip portion 34 having anti-slipping ribs 34A, to facilitate the handling of the dose setting member 16.

The dose setting member 16 may be made of polypropylene, polyester, polycarbonate, polypropylene, polyethylene, polyethylene terephthalate, acrylonitrile butadiene styrene, and preferably the dose setting member 16 may be made of polypropylene.

In the present example, the injection device 10 is configured so that the n^{th} portion is able to abut only against the n^{th} radial projection.

A dose setting method for pre-setting a dose of the injection device 10 before injection, comprising moving the dose setting member 16 in any one of the N positions so that an n^{th} radial projection axially face an n^{th} portion, is now described with reference to FIGS. 5A to 5G.

In FIG. 5A, the injection device 10 is not provided with the dose setting member 16. A reconstitution of an injection fluid, may be carried out by mixing a prefilled powder and a fluid. As an alternative, an injection fluid may be pumped/sucked from an external vial (not shown) containing the injection fluid while the reservoir 12A is initially empty. This may be carried out by moving the plunger rod in 14 a direction F2 (or proximal direction) extending along the axis X and oriented from the distal end DE toward the proximal end PE. Alternatively, the injection device 10 may be prefilled with the injection fluid and stored while prefilled.

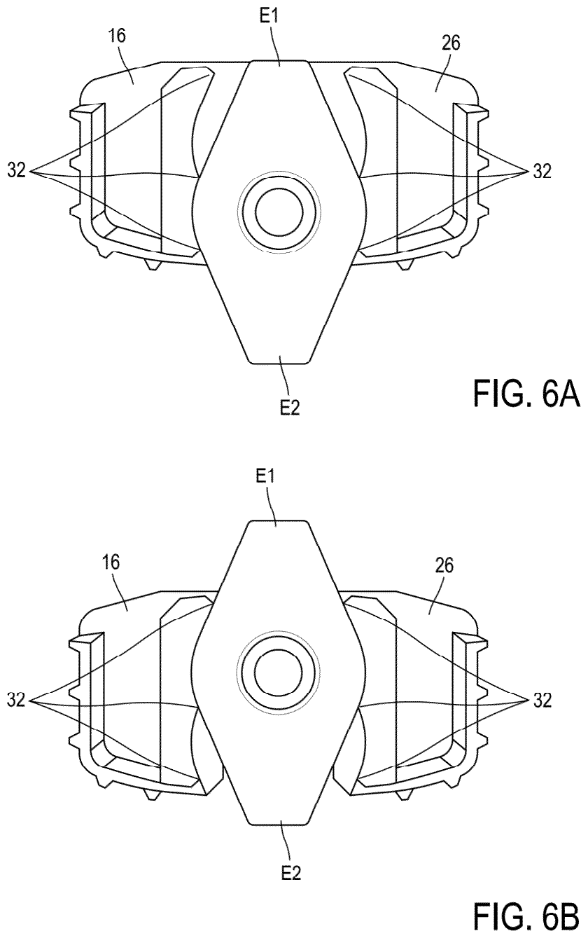

In FIG. 5B, the dose setting member 16 may be mounted onto the barrel 12 by radially inserting and sliding the flange 24 within the grooves 30 (see arrow I in FIGS. 5B and 6A) up to snap fit the edges E3, E4 with a last pair of reliefs 32, considered in the inserting direction (see FIG. 6A). In this position, the 1^{st} position of the dose setting member 16, the 1^{st} portion 16A axially faces the 1^{st} radial projection 14A. A first dose has been then pre-set. As seen in FIG. 6A, in the 1^{st} position, the tongue E2 extends through the opening 38.

In FIG. 5C, a first dose, in the present example a priming dose used to expel air bubbles potentially contained into the injection device, is carried out by moving the plunger rod 14 in the first direction F1, as shown by the arrow II, up to abutting the 1^{st} radial projection 14A against the 1^{st} portion 16A.

In FIG. 5D, the dose setting member 16 is then radially moved and slid along the second axis X2 (see arrow III), from the 1^{st} position to the 2^{nd} position of the dose setting member 16 in order to pre-set a second dose, corresponding to the first dose to be injected. In this position, the 2^{nd} portion 16B axially faces the 2^{nd} radial projection 14B. As shown in FIG. 6B, in such a position, the edges E3, E4 are snap fitted with the first pair of reliefs 32, considered in the inserting direction.

In FIG. 5E, a first injection is carried out by moving the plunger rod 14 in the first direction F1, as shown by the arrow IV, up to abutting the 2^{nd} radial projection 14B against the 2^{nd} portion 16B. It is noted that since the radius of the 1^{st} radial projection 14A is smaller than the minimal radial length RL2 of the 2^{nd} portion 16B, the 1^{st} radial projection does not cooperate with the 2^{nd} portion 16B.

In FIG. 5F, when the first injection is completed, the dose setting member 16 is radially moved and slid along the second axis X2 (see arrow V), up to be removed from the barrel 12. A 3^{rd} dose is then pre-set, corresponding to the second dose to be injected. In other words, the dose setting member is removed for setting the last dose.

In FIG. 5G, a second injection is carried out by moving the plunger rod 14 in the first direction F1, as shown by the arrow VI, up to the abutment of a portion of the plunger rod 14 with a portion of the barrel 12.

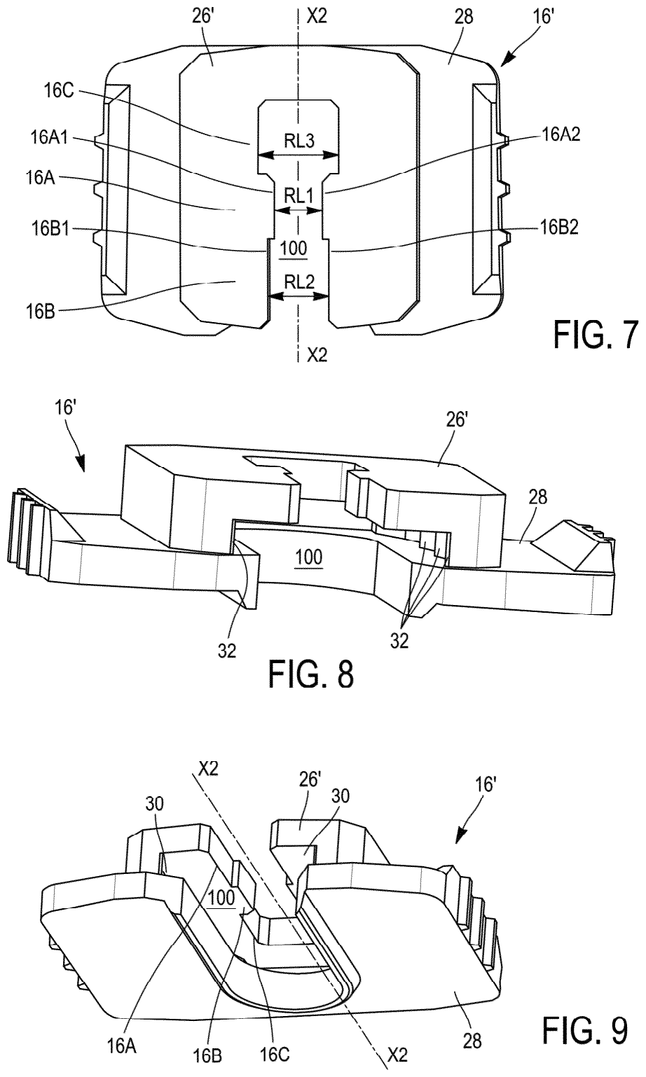

A first variant of the dose setting member 16′ is described with reference to FIGS. 7 to 9. The dose setting member 16′ is configured to adopt one N+1 position, wherein the plunger rod 14 is axially free relative to the dose setting member 16′.

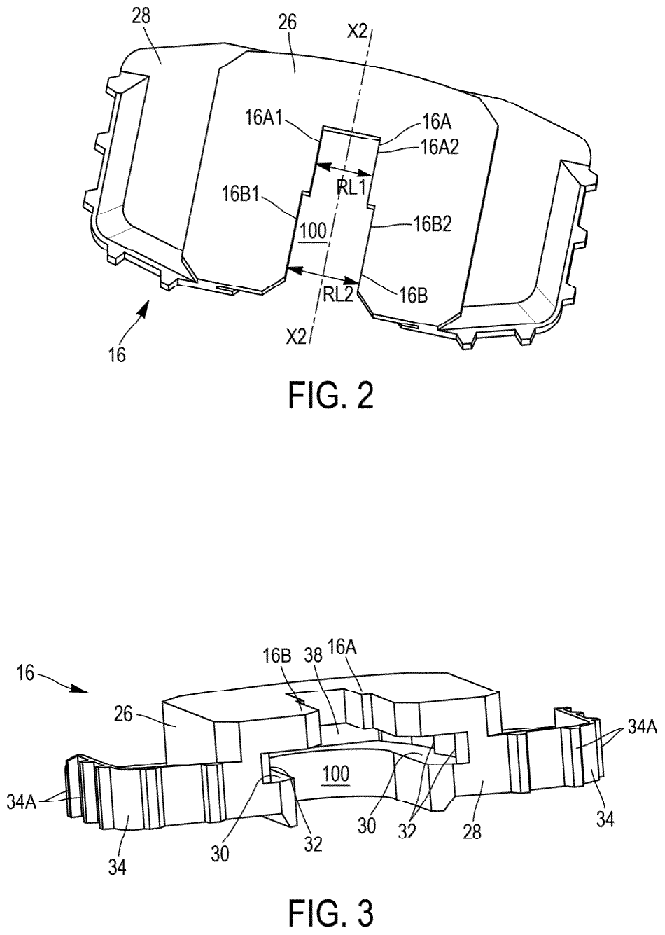

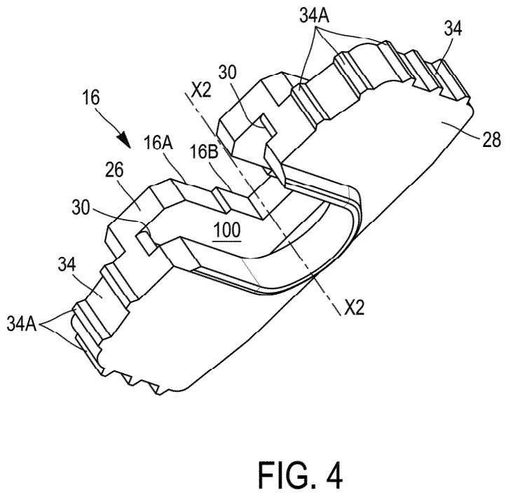

The dose setting member 16′ according to the first variant is similar to the dose setting member 16 of FIGS. 2 to 4, except with regard to the grip portion, which is arranged only on the two edges parallel to the second direction X2, and to a wide portion 16C of the notch 100 in the top portion 26′, which is not provided on the dose setting member 16 of FIGS. 2 to 4. The other parts are identical or very similar, are not described again and keep the same reference signs. It is note that the wording “wide” is used relative to the other portions, the minimal radial length of the wide portion 16C being greater than the radial length of the other portions 16A, 16C.

In the present example, due to the wide portion 16C the dose setting member 16′ defines an axial passage having a minimal radial length RL3 which is greater than the radius of the N^{th} radial projection 14B. The wide portion 16C corresponds to the N+1 position of the dose setting member 16′. In comparison with the dose setting member 16, the first variant 16′ is also provided with two additional reliefs 32 (i.e. an additional pair of facing reliefs 32), allowing a snap fitting with the flange 24 in the N+1 position. In the present example, the wide portion 16C is placed before the 1^{st} portion 16A, and corresponds to an initial position or a 0 position. The wide portion 16C is closer to the closed bottom of the “U” than the 1^{st} portion 16A. The 1^{st} portion 16A is axially disposed between the wide portion 16C and the 2^{nd} portion 16B.

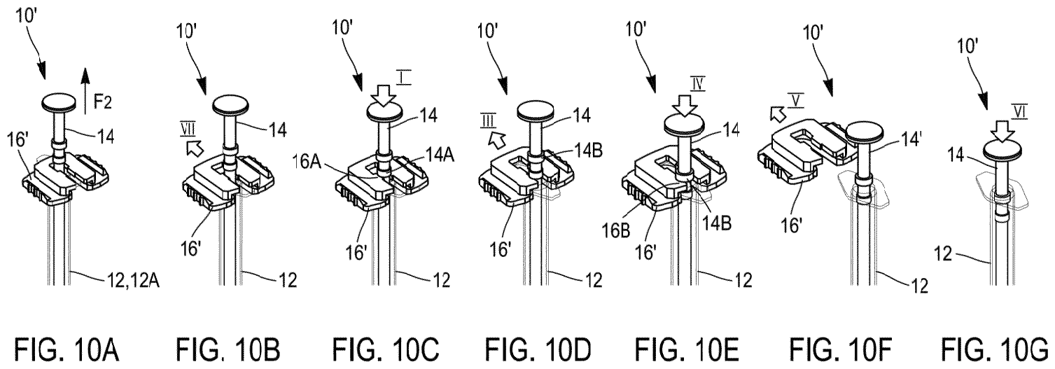

The injection device 10′ shown if FIGS. 10A to 10G corresponds to the injection device 10 wherein the dose setting member 16 is replaced by the dose setting member 16′ according to the first variant.

A dose setting method for pre-setting a dose of the injection device 10′ provided before injection, comprising moving the dose setting member 16′ in any one of the N positions so that an n^{th} radial projection axially face an n^{th} portion, is now described with reference to FIGS. 10A to 10G.

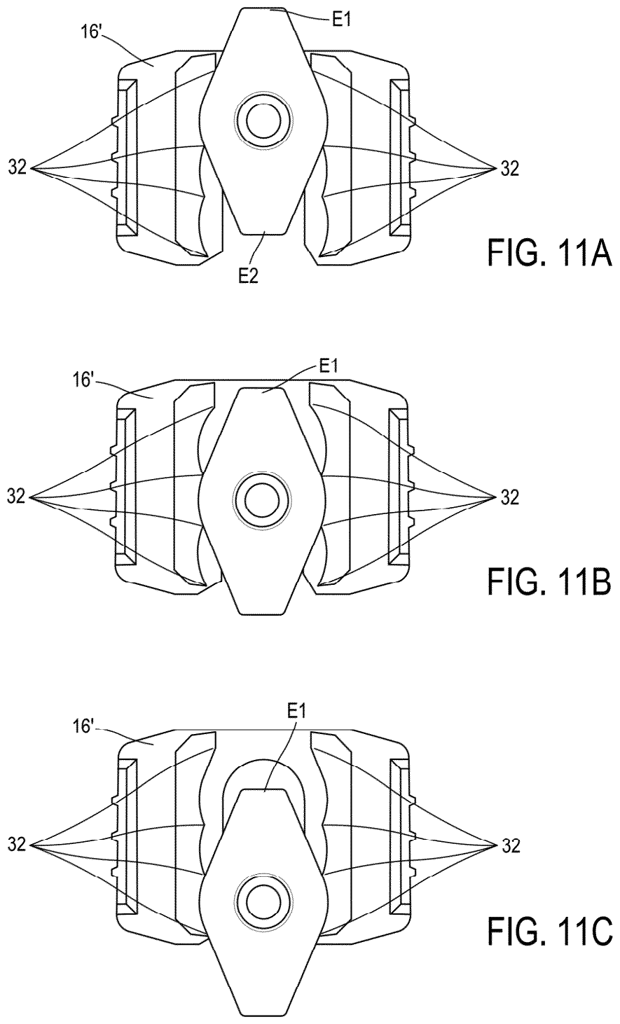

In FIG. 10A, the dose setting member 16′ is mounted (or pre-mounted in comparison with FIG. 5A) to the barrel 12, and place in the N+1 or initial position (see also the corresponding snap fitting configuration shown in FIG. 11A). In such a position, no portion of the dose setting member 16′ axially faces the radial projections 14A, 14B, and the plunger rod 14 is axially free relative to the dose setting member 16′. A reconstitution of an injection fluid, may be carried out by mixing a prefilled powder and a fluid. As an alternative, an injection fluid may be pumped/sucked from an external vial not shown while the reservoir 12A is initially empty. This may be carried out by moving the plunger rod in 14 a direction F2 extending along the axis X and oriented from the distal end DE toward the proximal end PE. Alternatively, the injection device 10 may be prefilled with the injection fluid and stored while prefilled. In other words, FIG. 10A correspond to a step of moving the dose setting member 16′ in the N+1 position before setting the first dose for filling the injection device 10′.

In FIG. 10B, the dose setting member 16′ may be radially moved and slid along the second axis X2 (see arrow VII) up to snap fit the edges E3, E4 with a second or intermediate pair of reliefs 32 (see FIG. 11B). In this position, the 1^{st} position of the dose setting member 16′, the 1^{st} portion 16A axially faces the 1^{st} radial projection 14A. A first dose has been then pre-set.

FIGS. 10C to 10G are strictly similar and strictly correspond to the steps of FIGS. 5C to 5G, respectively, and are not further described. In the same way, FIGS. 11B and 11C respectively corresponds to FIGS. 6A and 6B and are not further described.

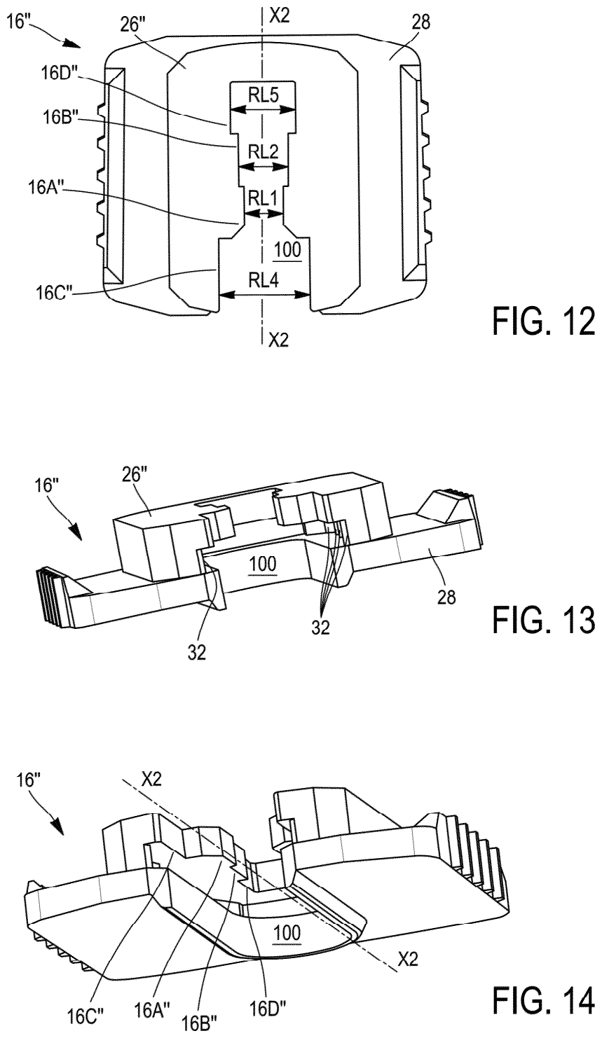

A second variant of the dose setting member 16″ is described with reference to FIGS. 12 to 14. The dose setting member 16″ is configured to adopt two N+1 positions, wherein the plunger rod 14 is axially free relative to the dose setting member 16″. The dose setting member 16″ may be un-removable, but not necessarily.

The dose setting member 16″ according to the second variant is similar to the dose setting member 16′ of the first variant, except with regard to arrangement of the portions on the top portion 26″. The other parts are identical or very similar, are not described again and keep the same reference signs.

The top portion 26″ of the dose setting member 16″ successively comprises, along the second axis X2, a first N+1 portion or wide portion 16C″, a 1^{st} portion 16A″, a 2^{nd} portion 16B″ and a second N+1 portion or wide portion 16D″, the first N+1 portion 16C″ being closer to the open top of the “U” than the second N+1 portion or wide portion 16D″. The second N+1 portion 16D″ is closer to the closed bottom of the “U” than the first N+1 portion 16C″.

In the present example, due to the first wide portion 16C″ the dose setting member 16″ defines a first axial passage having a minimal radial length RL4 which is greater than the radius of the N^{th} radial projection 14B. The first wide portion 16C″ corresponds to a first N+1 position of the dose setting member 16″. Due to the second wide portion 16D″, the dose setting member 16″ defines a second axial passage having a minimal radial length RL5 which is greater than the radius of the N^{th} radial projection 14B. The second wide portion 16D″ corresponds to a second N+1 position of the dose setting member 16″. In the present example RL4 is larger than RL5. However, RL4 may be smaller than RL5, or RL4 may be equal to RL5.

In comparison with the dose setting member 16, the second variant 16″ is also provided with four additional reliefs 32 (i.e. two additional pairs of facing reliefs 32), allowing a snap fitting with the flange 24 in the first and in the second N+1 positions.

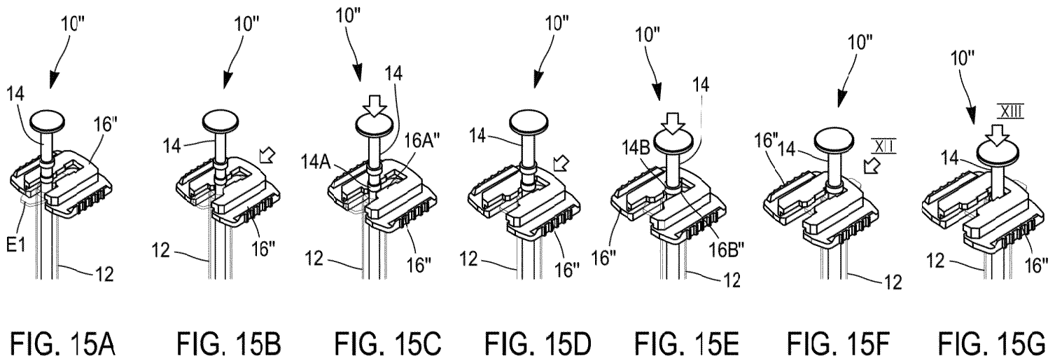

The injection device 10″ shown on FIGS. 15A to 15G correspond to the injection device 10 wherein the dose setting member 16 is replaced by the dose setting member 16″ according to the second variant.

A dose setting method for pre-setting a dose of the injection device 10″ provided before injection, comprising moving the dose setting member 16″ in any one of the N positions so that an n^{th} radial projection axially face an n^{th} portion, is now described with reference to FIGS. 15A to 15G.

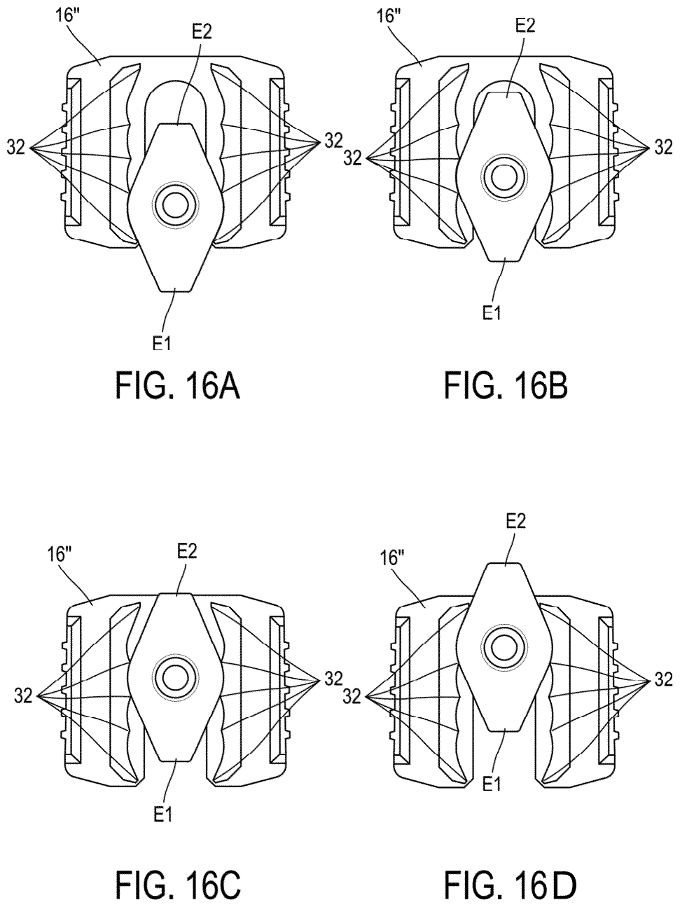

FIG. 15A to 15E respectively correspond to FIGS. 10A to 10E, except the radial movement of the dose setting member 16″ which is opposed due to the reverse order of the portions along the second axis X2. Therefore, FIGS. 15A to 15E are not further described. In the same way FIGS. 16A to 16C respectively corresponds to FIGS. 11A to 11C and are not further described.

In FIG. 15F, the dose setting member 16″ is moved and slid radially from the 2^{nd} position to the second N+1 position (see arrow XII). The corresponding snap fitting position is shown in FIG. 16D. This position is the last position of the dose setting member 16″, wherein a last dose is pre-set. In other words, FIG. 15 corresponds to moving the dose setting member 16″ in the second N+1 position after completion of the N^{th} dose to allow emptying the injection device 10″.

In FIG. 15G, a last injection is carried out by moving the plunger rod 14 in the first direction F1, as shown by the arrow XIII, up to the abutment of a portion of the plunger rod 14 with a portion of the barrel 12.

Although the present disclosure is described with reference to specific examples, it is to be understood that these examples are merely illustrative of the principles and applications of the present disclosure. In particular, individual characteristics of the various embodiments shown and/or mentioned may be combined in additional embodiments. Consequently, the description and the drawings should be considered in a sense that is illustrative rather than restrictive.

Additionally, all of the disclosed features of an apparatus may be transposed, alone or in combination, to a method and vice versa.

It is intended that the specification and the examples be considered as exemplary only, with a true scope of the invention being indicated by the following claims.

1. An injection device comprising:
a barrel extending along an axis and defining a reservoir, the barrel comprising a proximal end and a distal end having an outlet,
a plunger rod received within the barrel via the proximal end and configured to move along the axis relative to the barrel, the plunger rod comprising N radial projections successively disposed at different locations along the axis, N being an integer equal or greater than 2,
a dose setting member configured to be removably mounted onto the barrel, and having N portions configured to sequentially abut axially against the N radial projections, respectively, when moving the plunger rod axially in a first direction oriented from the proximal end toward the distal end,
the dose setting member being configured to be moved relative to the barrel between N positions, wherein the n^{th} radial projection axially faces the n^{th} portion (16A, 16B) in the n^{th} position, n being an integer lying between 1 and N.

2. The injection device according to claim 1, configured so that the n^{th} portion is able to abut only against the n^{th} radial projection.

3. The injection device according to claim 1, wherein the dose setting member is configured to adopt at least one N+1 position, wherein the plunger rod is axially free relative to the dose setting member.

4. The injection device according to claim 1, wherein the dose setting member is configured to be radially moved relative to the barrel between N positions.

5. The injection device according to claim 1, wherein the dose setting member comprises a notch extending radially and radially opened, considered when the dose setting member is mounted onto the barrel, and configured to receive the barrel.

6. The injection device according to claim 5, wherein the notch extends along a second axis, the N portions being successively disposed at different locations along the second axis.

7. The injection device according to claim 1, wherein a radial extension of the t^{th} radial projection is greater than a radial extension of the (t−1)^{th} radial projection and lower than a radial extension of the (t+1)^{th} radial projection, if any, t being an integer lying between 2 and N.

8. The injection device, according to claim 1, wherein each of the radial projections has an annular shape having a radius, the radius of the t^{th} radial projection being greater than the radius of the (t−1)^{th} radial projection, and lower than the radius of the (t+1)^{th} radial projection, if any, and each of the portions defines an axial passage having a minimal radial length, the minimal radial length (RL2) of the t^{th} portion (16B) being greater than minimal radial length of the (t−1)^{th} portion and lower than minimal radial length of the (t+1)^{th} portion, if any, t being an integer lying between 2 and N.

9. The injection device, according to claim 8, wherein the dose setting member is configured to adopt at least one N+1 position, wherein the plunger rod is axially free relative to the dose setting member, and wherein in the at least one N+1 position, the dose setting member defines an axial passage having a minimal radial length which is greater than the radius of the N^{th} radial projection.

10. The injection device according to claim 7, wherein the (t−1)^{th} radial projection is axially closer to the distal end than the t^{th} radial projection.

11. The injection device according to claim 1, wherein the barrel comprises a flange extending radially, the dose setting member being configured to be slidably mounted onto the flange.

12. The injection device according to claim 11, wherein the flange comprises a guiding portion configured to slidably guide the dose setting member, and a retaining portion configured to maintain the dose setting member in any of the N positions.

13. A dose setting method for pre-setting a dose of an injection device before injection, the dose setting method comprising providing an injection device according to claim 1, and moving the dose setting member in any one of the N positions so that a n^{th} radial projection axially face a n^{th} portion.

14. The dose setting method according to claim 13, wherein the dose setting member is removably mounted onto the barrel, the dose setting method comprising removing the dose setting member for setting the last dose.
